(19) United States
(12) Patent Application Publication
(10) Pub. No.: US 2007/0038223 A1
(43) Pub. Date: Feb. 15, 2007

(54) Computer-assisted knee replacement apparatus and method

(76) Inventors: Joel Marquart, Davie, FL (US); Marwan Sati, Mississauga (CA); Scott Illsley, Hollywood, FL (US); Louis K. Arata, Mentor, OH (US); Randall Hand, Pembroke Pines, FL (US); Arthur E. Quaid III, Hollywood, FL (US); Rony A. Abovitz, Hollywood, FL (US); Haniel Croitoru, Toronto (CA); James M. McKale, Syracuse, IN (US)

Correspondence Address: BOSE MCKINNEY & EVANS LLP;JAMES COLES, 135 N PENNSYLVANIA ST, SUITE 2700, INDIANAPOLIS, IN, 46204, US

(21) Appl. No.: 11/390,034

(22) Filed: Mar. 27, 2006

Related U.S. Application Data
(63) Continuation of application No. 11/199,557, filed on Aug. 8, 2005.
Application No. 11/199,557 is a continuation of application No. 11/007,623, filed on Dec. 6, 2004.
Application No. 11/007,623 is a continuation of application No. 10/772,139, filed on Feb. 4, 2004, now abandoned.
(60) Provisional application No. 60/445,078, filed on Feb. 4, 2003.

(51) Int. Cl. A61F 5/00

(52) U.S. Cl. 606/86

(57) ABSTRACT

A computer-assisted knee replacement apparatus and method comprises a knee replacement application for assisting, guiding, and planning a unicondylar knee replacement procedure. The apparatus and method cooperates with a tracking system to determine implant sizing and location. The apparatus and method also cooperates with the tracking system to determine required tibial and femoral preparation corresponding to the implant size and location and provides real-time monitoring of the tibial and femoral surface preparation procedures.

This patent application is a continuation of U.S. patent application Ser. No. 11/007,623, entitled “Computer Assisted Knee Replacement Apparatus and Method,” filed Dec. 6, 2004 which is a continuation of U.S. patent application Ser. No. 10/772,139, entitled “Computer-Assisted Knee Replacement Apparatus and Method,” filed Feb. 4, 2004; and claims the benefit of U.S. provisional patent application Ser. No. 60/445,078, entitled “Computer-Assisted Knee Replacement Apparatus and Method,” filed Feb. 4, 2003, the disclosure of which is incorporated herein by reference. This application relates to the following U.S. provisional patent applications Ser. No. 60/444,824, entitled “Interactive Computer-Assisted Surgery System and Method”; Ser. No. 60/444,975, entitled “System and Method for Providing Computer Assistance With Spinal Fixation Procedures”; Ser. No. 60/444,989, entitled “Computer-Assisted External Fixation Apparatus and Method”; Ser. No. 60/444,988, entitled “Computer-Assisted Knee Replacement Apparatus and Method”; Ser. No. 60/445,002, entitled “Method and Apparatus for Computer Assistance With Total Hip Replacement Procedure”; Ser. No. 60/445,001, entitled “Method and Apparatus for Computer Assistance With Intramedullary Nail Procedure”; and Ser. No. 60/319,924, entitled “Portable, Low-Profile Integrated Computer, Screen and Keyboard for Computer Surgery Applications”; each of which was filed on Feb. 4, 2003 and is incorporated herein by reference. This application also relates to the following applications: U.S. patent application Ser. No. 10/772,083, entitled “Interactive Computer-Assisted Surgery System and Method”; U.S. patent application Ser. No. 10/771,85, entitled “System and Method for Providing Computer Assistance With Spinal Fixation Procedures”; U.S. patent application Ser. No. 10/772,142, entitled Computer-Assisted External Fixation Apparatus and Method“; U.S. patent application Ser. No. 10/772,085, entitled “Computer-Assisted Knee Replacement Apparatus and Method”; U.S. patent application Ser. No. 10/772,092, entitled “Method and Apparatus for Computer Assistance With Total Hip Replacement Procedure”; U.S. patent application Ser. No. 10/771,851, entitled “Method and Apparatus for Computer Assistance With Intramedullary Nail Procedure”; and U.S. patent application Ser. No. 10/772,137, entitled “Portable Low-Profile Integrated Computer, Screen and Keyboard for Computer Surgery Applications”; each of which was filed on Feb. 4, 2004 and is incorporated herein by reference.

### TECHNICAL FIELD OF THE INVENTION

The present invention relates generally to the field of computer-assisted surgery systems and methods and, more particularly, to a computer-assisted knee replacement apparatus and method.

### BACKGROUND OF THE INVENTION

Image-based surgical navigation systems display the positions of surgical tools with respect to preoperative (prior to surgery) or intraoperative (during surgery) image datasets. Two and three dimensional image data sets are used, as well as time-variant images data (i.e. multiple data sets take at different times). Types of data sets that are primarily used include two-dimensional fluoroscopic images and three-dimensional data sets include magnetic resonance imaging (MRI) scans, computer tomography (CT) scans, positron emission tomography (PET) scans, and angiographic data. Intraoperative images are typically fluoroscopic, as a C-arm fluoroscope is relatively easily positioned with respect to patient and does not require that a patient be moved. Other types of imaging modalities require extensive patient movement and thus are typically used only for preoperative and post-operative imaging.

The most popular navigation systems make use of a tracking or localizing system to track tools, instruments and patients during surgery. These systems locate in predefined coordinate space specially recognizable markers or elements that are attached or affixed to, or possibly inherently a part of, an object such as an instrument or a patient. The elements can take several forms, including those that can be located using optical (or visual), magnetic, or acoustical methods. Furthermore, at least in the case of optical or visual systems, the location of an object's position may be based on intrinsic features or landmarks that, in effect, function as recognizable elements. The elements will have a known, geometrical arrangement with respect to, typically, an end point and/or axis of the instrument. Thus, objects can be recognized at least in part from the geometry of the elements (assuming that the geometry is unique), and the orientation of the axis and location of endpoint within a frame of reference deduced from the positions of the elements.

A typical optical tracking system functions primarily in the infrared range. They usually include a stationary stereo camera pair that is focused around the area of interest and sensitive to infrared radiation. Elements emit infrared radiation, either actively or passively. An example of an active element is a light emitting diode (LED). An example of a passive element is a reflective element, such as ball-shaped element with a surface that reflects incident infrared radiation. Passive systems require an infrared radiation source to illuminate the area of focus. A magnetic system may have a stationary field generator that emits a magnetic field that is sensed by small coils integrated into the tracked tools.

Most computer-assisted surgery (CAS) systems are capable of continuously tracking, in effect, the position of tools (sometimes also called instruments). With knowledge of the position of the relationship between the tool and the patient and the patient and an image data sets, a system is able to continually superimpose a representation of the tool on the image in the same relationship to the anatomy in the image as the relationship of the actual tool to the patient's anatomy. To obtain these relationships, the coordinate system of the image data set must be registered to the relevant anatomy of the actual patient and portions of the of the patient's anatomy in the coordinate system of the tracking system. There are several known registration methods.

In CAS systems that are capable of using two-dimensional image data sets, multiple images are usually taken from different angles and registered to each other so that a representation of the tool or other object (which can be real or virtual) can be, in effect, projected into each image. As the position of the object changes in three-dimensional space, its projection into each image is simultaneously updated. In order to register two or more two-dimensional data images together, the images are acquired with what is called a registration phantom in the field of view of the image device. In the case of a two-dimensional fluoroscopic images, the phantom is a radio-translucent body holding radio-opaque fiducials having a known geometric relationship. Knowing the actual position of the fiducials in three-dimensional space when each of the images are taken permits determination of a relationship between the position of the fiducials and their respective shadows in each of the images. This relationship can then be used to create a transform for mapping between points in three-dimensional space and each of the images. By knowing the positions of the fiducials with respect to the tracking system's frame of reference, the relative positions of tracked tools with respect to the patient's anatomy can be accurately indicated in each of the images, presuming the patient does not move after the image is acquired, or that the relevant portions of the patient's anatomy are tracked. A more detailed explanation of registration of fluoroscopic images and coordination of representations of objects in patient space superimposed in the images is found in U.S. Pat. No. 6,198,794 of Peshkin, et al., entitled “Apparatus and method for planning a stereotactic surgical procedure using coordinated fluoroscopy.”

### SUMMARY OF THE INVENTION

The invention is generally directed to improved computer-implemented methods and apparatus for further reducing the invasiveness of surgical procedures, eliminating or reducing the need for external fixtures in certain surgical procedures, and/or improving the precision and/or consistency of surgical procedures. The invention finds particular advantage in orthopedic procedures involving implantation of devices, though it may also be used in connection with other types of surgical procedures.

The computer-assisted knee replacement apparatus and method provide a series of graphical user interfaces and corresponding procedural guidelines for performing a knee replacement procedure. For example, according to one embodiment, a computer-assisted knee replacement application comprises a series of graphical user interfaces and corresponding guidelines and instructions for performing a unicondular knee replacement procedure. In this embodiment, the knee replacement application cooperates with a tracking system to provide real-time evaluation and monitoring of knee modifications to increase the accuracy of knee implant positioning and implantation. For example, the knee replacement application cooperates with the tracking system to monitor the position of burring tools during burring operations and provides real-time indications of the burring procedure to accommodate a particular knee implant. In this embodiment, the knee replacement application also cooperates with the tracking system to acquire kinematic data associated with movement of the knee to increase the accuracy of knee implant placement. The knee replacement application also provides sizing information for the implant based on data acquired using the tracking system.

### BRIEF DESCRIPTION OF THE DRAWINGS

For a more complete understanding of the present invention and the advantages thereof, reference is now made to the following descriptions taken in connection with the accompanying drawings in which:

FIG. 1 is a block diagram illustrating an exemplary computer-assisted surgery system;

FIG. 2 is a flow chart of basic steps of an application program for assisting with or guiding the planning of, and navigation during, a unicondylar knee replacement procedure; and

FIGS. 3-11 are representative screen images of graphical user interface pages generated and displayed by the application program of FIG. 2.

### DETAILED DESCRIPTION OF THE DRAWINGS

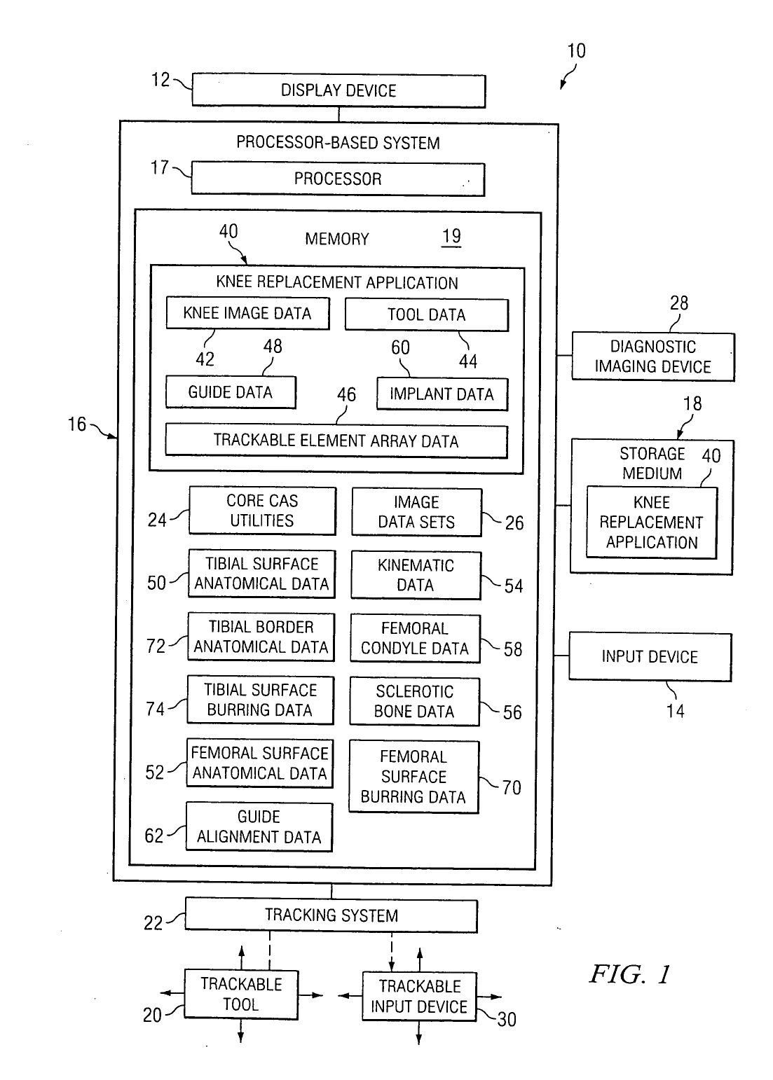

The preferred embodiments of the present invention and the advantages thereof are best understood by referring to FIGS. 1-11 of the drawings, like numerals being used for like and corresponding parts of the various drawings.

FIG. 1 is a block diagram of an exemplary computer-assisted surgery (CAS) system 10. CAS system 10 comprises a display device 12, an input device 14, and a processor-based system 16, for example a computer. Display device 12 may be any display device now known or later developed for displaying two-dimensional and/or three-dimensional diagnostic images, for example, a monitor, a touch screen, a wearable display, a projection display, a head-mounted display, stereoscopic views, a holographic display, a display device capable of displaying image(s) projected from an image projecting device, for example a projector, and/or the like. Input device 14 may be any input device now known or later developed, for example, a keyboard, a mouse, a trackball, a trackable probe, and/or the like. The processor-based system 16 is preferably programmable and includes one or more processors 17, working memory 19 for temporary program and data storage that will be used primarily by the processor, and storage for programs and data, preferably persistent, such as a disk drive. Removable media storage medium 18 can also be used to store programs and/or data transferred to or from the processor-based system 16. The storage medium 18 may include a floppy disk, an optical disc, or any other type of storage medium now known or later developed.

Tracking system 22 continuously determines, or tracks, the position of one or more trackable elements disposed on, incorporated into, or inherently a part of surgical instruments or tools 20 with respect to a three-dimensional coordinate frame of reference. With information from the tracking system 22 on the location of the trackable elements, CAS system 10 is programmed to be able to determine the three-dimensional coordinates of an endpoint or tip of a tool 20 and, optionally, its primary axis using predefined or known (e.g. from calibration) geometrical relationships between trackable elements on the tool and the endpoint and/or axis of the tool 20. A patient, or portions of the patient's anatomy, can also be tracked by attachment of arrays of trackable elements.

The CAS system 10 can be used for both planning surgical procedures (including planning during surgery) and for navigation. It is therefore preferably programmed with software for providing basic image guided surgery functions, including those necessary for determining the position of the tip and axis of instruments and for registering a patient and preoperative and/or intraoperative diagnostic image data sets to the coordinate system of the tracking system. The programmed instructions for these functions are indicated as core CAS utilities 24. These capabilities allow the relationship of a tracked instrunent to a patient to be displayed and constantly updated in real time by the CAS system 10 overlaying a representation of the tracked instrument on one or more graphical images of the patient's anatomy on display device 12. The graphical images may be a virtual representation of the patient's anatomy or may be constructed from one or more stored image data sets 26 acquired from a diagnostic imaging device 28. The imaging device may be a fluoroscope, such as a C-arm fluoroscope, capable of being positioned around a patient laying on an operating table. It may also be a MR, CT or other type of imaging device in the room or permanently located elsewhere. Where more than one image is shown, as when multiple fluoroscopic images are simultaneously displayed of display device 12, the representation of the tracked instrument or tool is coordinated between the different images. However, CAS system 10 can be used in some procedures without the diagnostic image data sets, with only the patient being registered. Thus, the CAS system 10 may need not to support the use diagnostic images in some applications—i.e., an imageless application.

Furthermore, as disclosed herein, the CAS system 10 may be used to run application-specific programs that are directed to assisting a surgeon with planning and/or navigation during specific types of procedures. For example, the application programs may display predefined pages or images corresponding to specific steps or stages of a surgical procedure. At a particular stage or part of a program, a surgeon may be automatically prompted to perform certain tasks or to define or enter specific data that will permit, for example, the program to determine and display appropriate placement and alignment of instrumentation or implants or provide feedback to the surgeon. Other pages may be set up to display diagnostic images for navigation and to provide certain data that is calculated by the system for feedback to the surgeon. Instead of or in addition to using visual means, the CAS system 10 could also communicate information in ways, including using audibly (e.g. using voice synthesis) and tactilely, such as by using a haptic interface type of device. For example, in addition to indicating visually a trajectory for a drill or saw on the screen, the CAS system 10 may feedback to a surgeon information whether he is nearing some object or is on course with a audible sound or by application of a force or other tactile sensation to the surgeon's hand.

To further reduce the burden on the surgeon, the program may automatically detect the stage of the procedure by recognizing the instrument picked up by a surgeon and move immediately to the part of the program in which that tool is used. Application data generated or used by the application may also be stored in processor-based system 16.

Various types of user input methods can be used to improve ease of use of the CAS system 10 during surgery. One example is the use the use of speech recognition to permit a doctor to speak a command. Another example is the use of a tracked object to sense a gesture by a surgeon, which is interpreted as an input to the CAS system 10. The meaning of the gesture could further depend on the state of the CAS system 10 or the current step in an application process executing on the CAS system 10. Again, as an example, a gesture may instruct the CAS system 10 to capture the current position of the object. One way of detecting a gesture is to occlude temporarily one or more of the trackable elements on the tracked object (e.g. a probe) for a period of time, causing loss of the CAS system's 10 ability to track the object. A temporary visual occlusion of a certain length (or within a certain range of time), coupled with the tracked object being in the same position before the occlusion and after the occlusion, would be interpreted as an input gesture. A visual or audible indicator that a gesture has been recognized could be used to provide feedback to the surgeon.

Yet another example of such an input method is the use of tracking system 22 in combination with one or more trackable data input devices 30. Defined with respect to the trackable input device 30 are one or more defined input areas, which can be two-dimensional or three-dimensional. These defined input areas are visually indicated on the trackable input device 30 so that a surgeon can see them. For example, the input areas may be visually defined on an object by representations of buttons, numbers, letters, words, slides and/or other conventional input devices. The geometric relationship between each defined input area and the trackable input device 30 is known and stored in processor-based system 16. Thus, the processor 17 can determine when another trackable object touches or is in close proximity a defined input area and recognize it as an indication of a user input to the processor based system 16. For example, when a tip of a tracked pointer is brought into close proximity to one of the defined input areas, the processor-based system 16 will recognize the tool near the defined input area and treat it as a user input associated with that defined input area. Preferably, representations on the trackable user input correspond user input selections (e.g. buttons) on a graphical user interface on display device 12. The trackable input device 30 may be formed on the surface of any type of trackable device, including devices used for other purposes. In a preferred embodiment, representations of user input functions for graphical user interface are visually defined on a rear, flat surface of a base of a tool calibrator.

Processor-based system 16 is, in one example, a programmable computer that is programmed to execute only when single-use or multiple-use software is loaded from, for example, removable media 18. The software would include, for example the application program for use with a specific type of procedure. The application program can be sold bundled with disposable instruments specifically intended for the procedure. The application program would be loaded into the processor-based system 16 and stored there for use during one (or a defined number) of procedures before being disabled. Thus, the application program need not be distributed with the CAS system 10. Furthermore, application programs can be designed to work with specific tools and implants and distributed with those tools and implants. Preferably, also, the most current core CAS utilities 24 may also be stored with the application program. If the core CAS utilities 24 on the processor-based system 16 are outdated, they can be replaced with the most current utilities.

In FIG. 1, the application program comprises a unicondylar knee replacement application 40 for assisting with, planning, and guiding a unicondylar or Repecci knee replacement procedure. The knee replacement application 40 provides a series of displayable images and corresponding instructions or guidelines for performing the knee replacement procedure. The knee replacement application 40 may be loaded into the processor-based system 16 from the media storage device 18. Processor-based system 16 may then execute the knee replacement application 40 solely from memory 19 or portions of the application 40 may be accessed and executed from both memory 19 and the storage medium 18.

Briefly, knee replacement application 40 cooperates with tracking system 22 to acquire static and/or kinematic data associated with a patient or subject to increase the accuracy of knee implant sizing, knee implant placement, and knee modifications to accommodate the knee implants. For example, using trackable tools 20, tracking system 22 tracks the location and position of tools 20 using trackable element arrays secured or otherwise coupled to tools 20. Trackable element arrays are also placed or coupled to portions of the subject in relation to the knee. For example, a trackable element array may be secured or otherwise coupled to the femur and the tibia/fibula of the subject. The tracking system 22 may then calibrate or register tools 20 with the trackable element arrays coupled to the subject. Thus, in operation, the knee replacement application 40 cooperates with the tracking system 22 to acquire static data associated with the physical characteristics of the subject's knee and kinematic data associated with movement of the tibia/fibula relative to the femur of the subject. Using the acquired static and kinematic data, the knee replacement application 40 determines a knee implant size, the modifications to be made to the femur and/or tibia to accommodate the knee implants, and the locations of the implants in the femur and/or tibia corresponding to various characteristics of the femur and/or tibia of the subject.

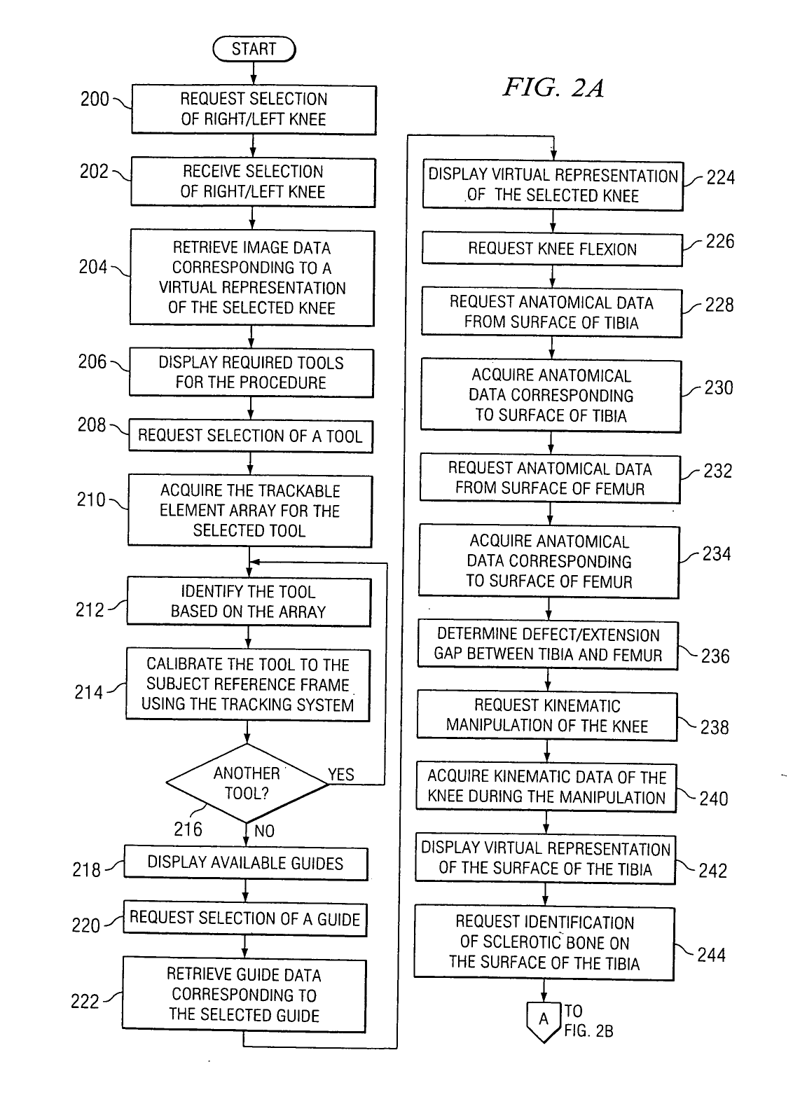

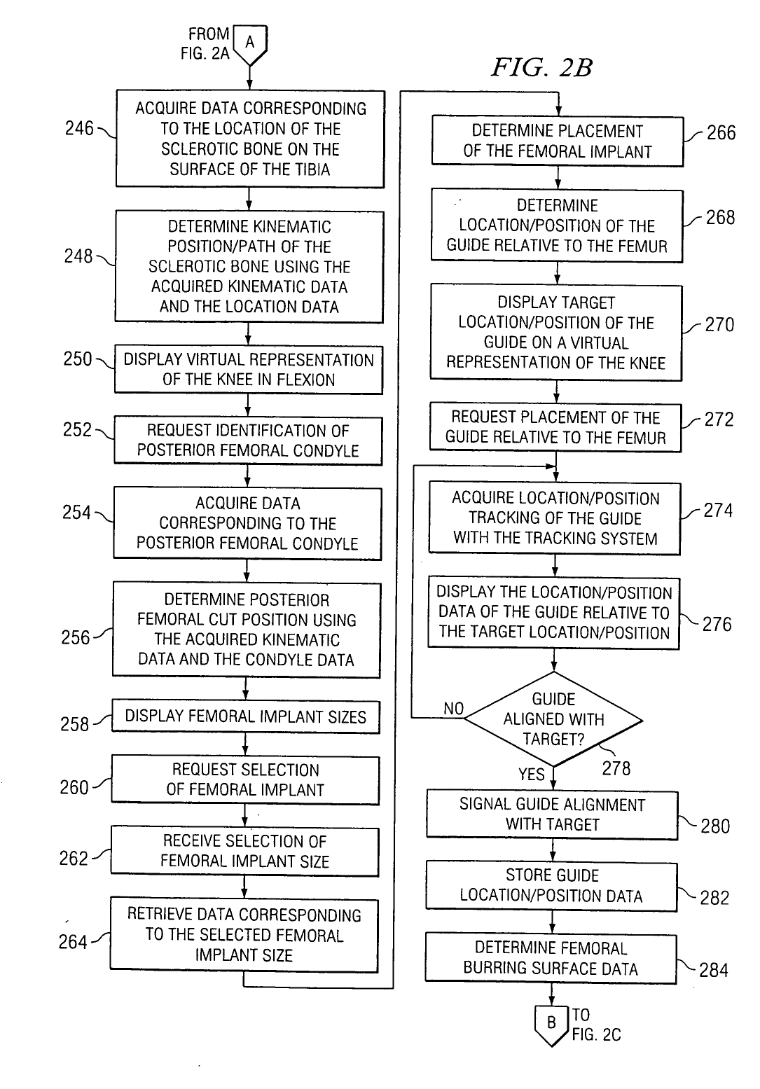

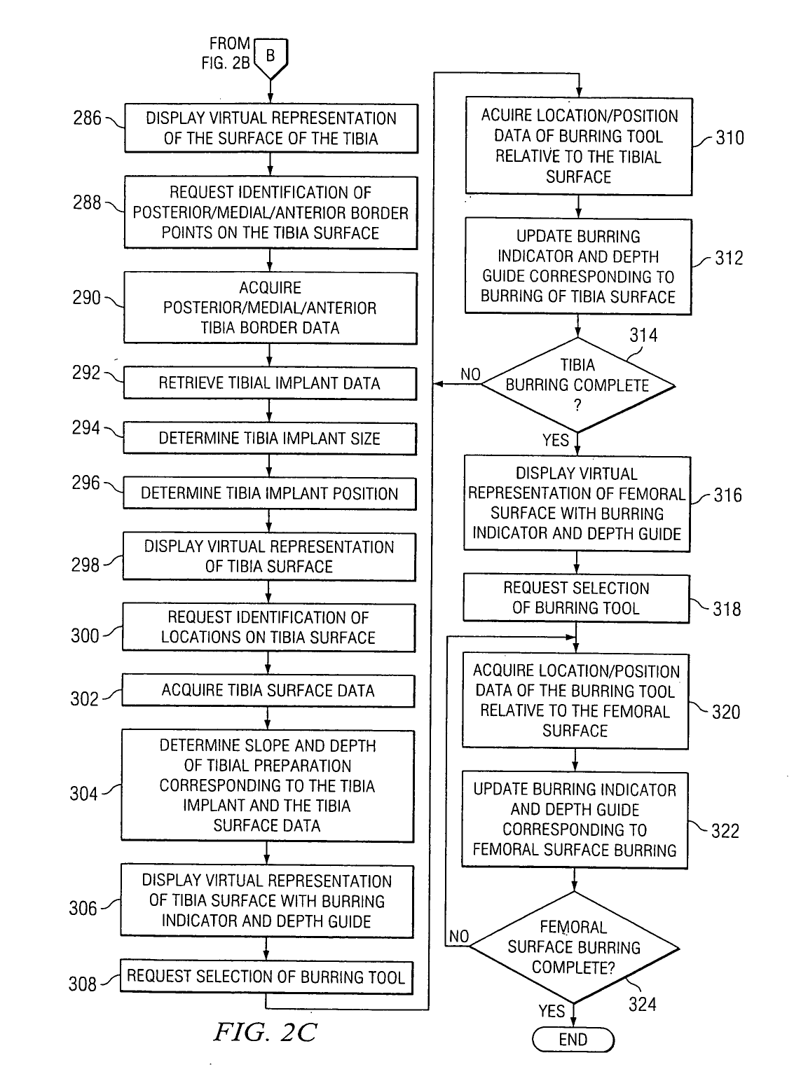

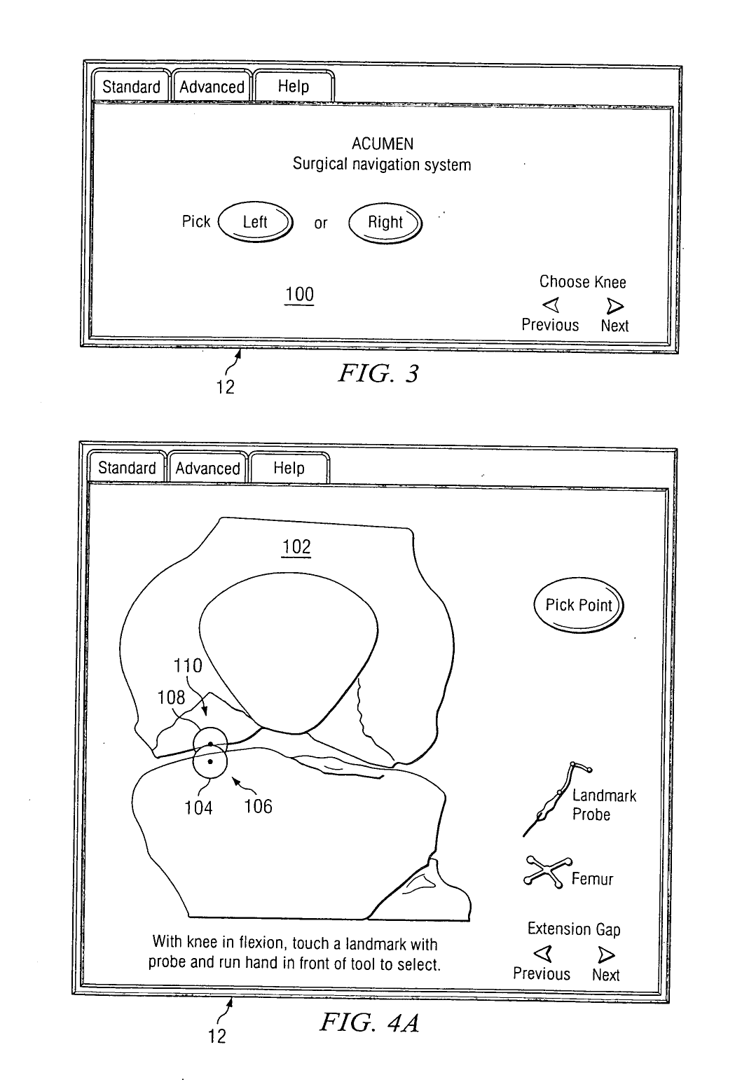

FIG. 2 is a flowchart illustrating an exemplary embodiment of a series of steps of the knee replacement application 40 in accordance with the present invention. The method begins at step 200, where the knee replacement application 40 requests selection of either a right or left knee to which the procedure will be performed. The request may be displayed on display device 12 to accommodate selection of either the right or left knee by a touch screen associated with display device 12 or may be otherwise selected using input device 14. For example, FIG. 3 illustrates a graphical user interface image 100 requesting the selection of either a left or right knee for performing the procedure, and at step 202, the knee replacement application 40 receives a selection of either the right or left knee. The knee replacement application 40 may output information, such as requests or instructions, to the user audibly or visually, such as with display device 12. The knee replacement application 40 may also provide output information to the user haptically. For example, as will be described in greater detail below, the knee replacement application 40 provides alignment and other types of information in connection with the knee replacement procedure corresponding to trackable tools 20, resection guides, and other devices. The knee replacement application 40 may be configured to provide haptic output to the user when performing these alignment and other procedural steps. At step 204, the knee replacement application 40 retrieves image data 42 having image information associated with a virtual representation of the selected knee. For example, the image data 42 may comprise image information associated with general bone and/or tissue structures of a knee such that a virtual representation of a knee may be displayed onto display device 12.

At step 206, the knee replacement application 40 retrieves tool data 44 to display a listing of required tools 20 for the procedure. At step 208, the replacement application 40 requests that the user select one of the tools 20. At step 210, the tracking system 22 acquires the trackable element array of the selected tool as the tool 20 enters an input area of the tracking system 22. At step 212, the knee replacement application 40 retrieves or accesses trackable element array data 46 and identifies the selected tool 20 based on the array data 46. For example, each trackable element array may be geometrically configured such that each geometrical array is associated with a particular tool 20 or a particular location on the subject. Thus, the knee replacement application 40 and tracking system 22 may automatically identify and associate each trackable element array with a corresponding tool 20 or subject position. At step 214, tracking system 22 calibrates the tool 20 to the subject reference frame. At decisional step 216, a determination is made whether another tool 20 requires selection and calibration. If another tool 20 requires selection and calibration, the method returns to step 212. If no other tools 20 require selection and calibration, the method proceeds to step 218.

At step 218, knee replacement application 40 displays on display device 12 available guides for the procedure. For example, in a unicondylar knee replacement procedure, a guide may be used to locate resection lines or planes, burring locations, implant keel locations, or implant mounting holes or channels to be made in either the femur and/or tibia. At step 220, the knee replacement application 40 requests selection of a particular guide by the user. At step 222, the knee replacement application 40 retrieves guide data 48 corresponding to the selected guide. For example, the guide data 48 may comprise information associated with the geometrical characteristics of the selected guide such that locating and/or positioning of the guide relative to the knee of the subject may be accurately determined based on static and/or kinematic data acquired by tracking system 22. As described above, the guide is also coupled to a trackable element array such that the tracking system 22 and knee replacement application 40 may locate and guide the positioning of the guide relative to the subject.

At step 224, the knee replacement application 40 displays a virtual representation 102 of the selected knee on display device 12 as illustrated in FIG. 4A. At step 226, the knee replacement application 40 requests flexion of the selected knee of the subject. At step 228, the knee replacement application 40 requests acquisition of anatomical data 50 from a surface of the tibia of the subject. For example, as best illustrated in FIG. 4A, the knee replacement application 40 may indicate a particular location 104 of the tibial surface 106 on the virtual representation 102 of the knee displayed on display device 12 and request that the user touch or locate the indicated tibial surface 106 of the subject using a trackable tool 20. At step 230, the knee replacement application 40 acquires the requested anatomical data 50 corresponding to the surface 106 of the tibia using tracking system 22. At step 232, the knee replacement application 40 requests anatomical data 52 corresponding to a surface of the femur of the subject. For example, as best illustrated in FIG. 4A, the knee replacement application 40 may indicate a particular location 108 on the femoral surface 110 on the virtual representation 102 of the knee displayed on display device 12 and request that the user touch or select the indicated femoral location 108 of the subject using a trackable tool 20. At step 234, the knee replacement application 40 acquires the requested anatomical data 52 corresponding to the surface 110 of the femur using tracking system 22. At step 236, the knee replacement application 40 calculates or determines an extension gap or defect gap between the tibia and the femur of the subject using the acquired tibia and femur anatomical data 50 and 52. Alternatively, or additionally, replacement application 40 may request the user to select or otherwise acquire an accuracy landmark(s) on the femur and/or tibia of the subject that can be readily re-acquired using trackable tool 20, as best illustrated in FIG. 4B, such that the selected landmark(s) may be subsequently used during the procedure for accuracy verification. Thus, by re-acquiring the landmark(s) using trackable tool 20, the user may determine if a tracking reference array on the subject has moved.

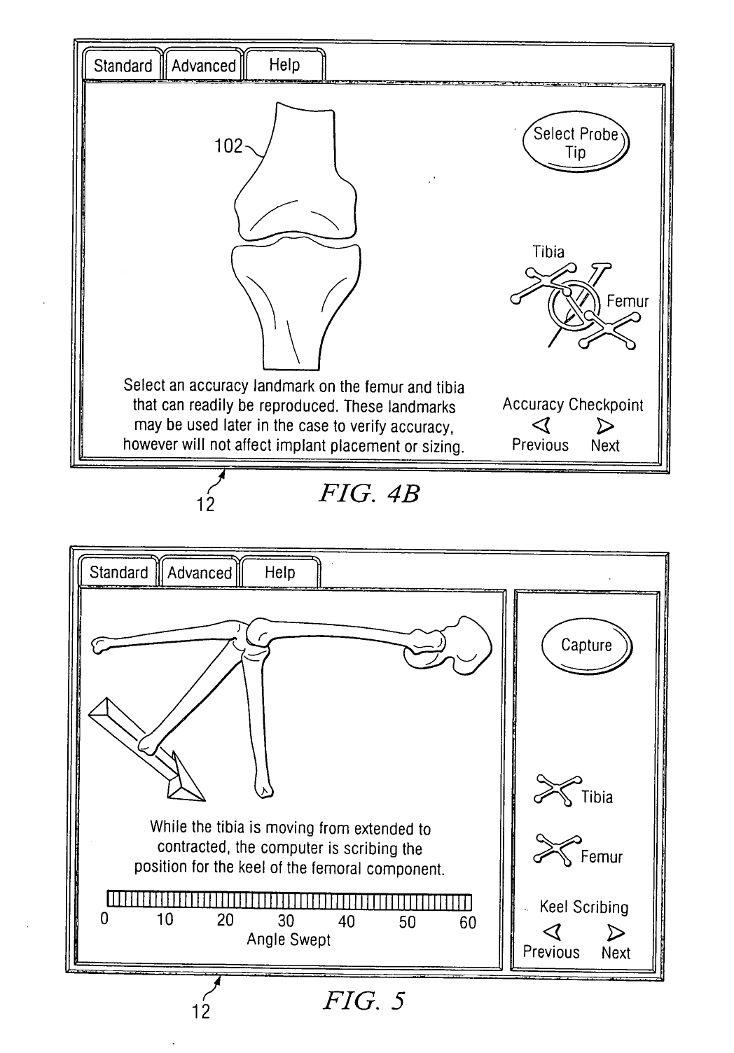

At step 238, the knee replacement application 40 requests kinematic manipulation of the selected knee. For example, as best illustrated in FIG. 5, the knee replacement application 40 may instruct the user to flex and/or extend the tibia of the subject relative to the femur of the subject. At step 240, the tracking system 22 acquires kinematic data 54 of the tibial movement during the kinematic manipulation of the tibia. For example, the kinematic data 54 may be acquired using the trackable element arrays coupled to the femur and the tibia/fibula of the subject. As will be described in greater detail below, the knee replacement application 40 uses the kinematic data 54 to determine a location for a keel of a femoral implant corresponding to sclerotic bone structure of the tibia.

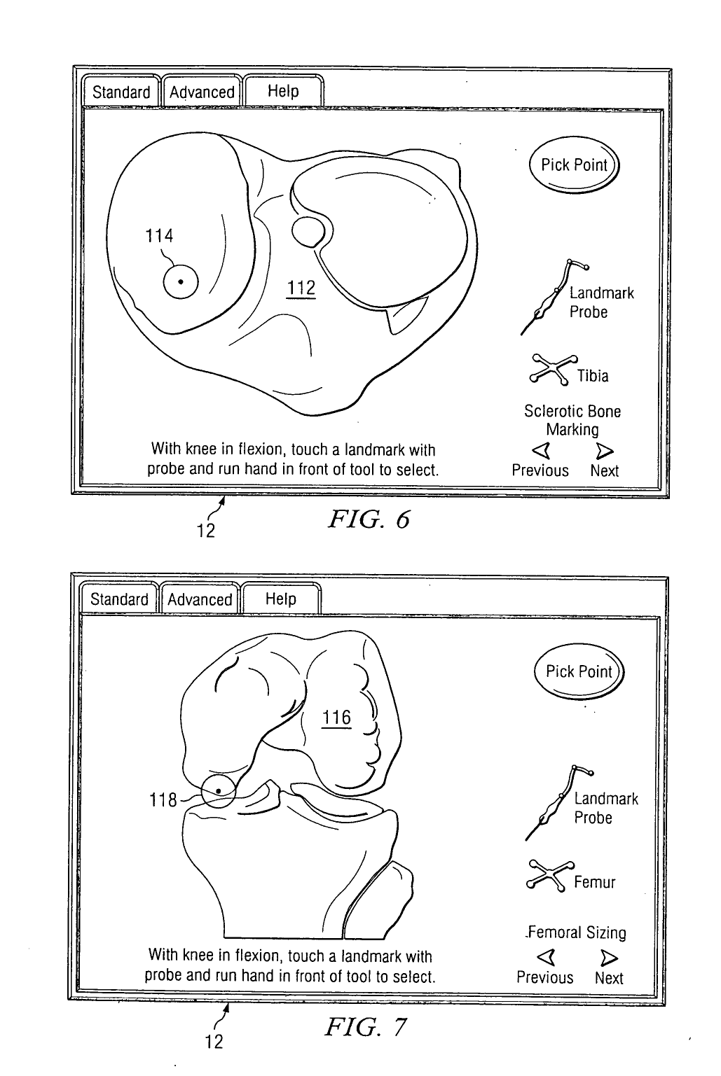

At step 242, the knee replacement application 40 displays on display device 12 a virtual representation 112 of the surface of the tibia, as best illustrated in FIG. 6. At step 244, the knee replacement application 40 requests identification or selection of the sclerotic bone structure on the surface of the tibia. For example, as illustrated in FIG. 6, the knee replacement application 40 may identify a general area 114 on the surface of the tibia generally associated with the sclerotic bone structure. The user may then identify and select the sclerotic bone location on the tibia of the subject using a trackable tool 20. At step 246, the knee replacement application 40 acquires data 56 corresponding to the location 114 of the sclerotic bone on the surface of the tibia using tracking system 22. At step 248, the knee replacement application 40 determines the kinematic position or path of the sclerotic bone of the tibia relative to the femur using the sclerotic bone data 56 acquired at step 246 and the kinematic data 54 acquired at step 240. Thus, by determining the kinematic position or path of the sclerotic bone of the tibia relative to the femur, the knee replacement application 40 automatically determines a location and orientation of a femur implant relative to the location of the sclerotic bone of the tibia of the subject.

At step 250, the knee replacement application 40 displays a virtual representation 116 of the selected knee in flexion and requests manipulation of the knee into a flexed position, as best illustrated in FIG. 7. At step 252, the knee replacement application 40 requests identification of the posterior femoral condyle of the femur of the subject. For example, the posterior femoral condyle may be identified by the user by indicating or touching the posterior femoral condyle at a general location 118 indicated by knee replacement application 40 on the virtual representation 116 displayed on display device 12 using a trackable tool 20. At step 254, the knee replacement application 40 acquires data 58 corresponding to the posterior femoral condyle using tracking system 22. At step 256, the knee replacement application 40 determines the posterior femoral resection position or plane relative to the femur using the condyle data 58 acquired at step 254 and the kinematic data 54 acquired at step 238 which correlates the implant location to the sclerotic bone of the tibia.

At step 258, the knee replacement application 40 displays available femoral implant sizes on display device 12, indicated generally by 120 as illustrated in FIG. 8. At step 260, the knee replacement application 40 requests selection of a particular femoral implant size by the user. At step 262, the knee replacement application 40 receives a selection of a particular femoral implant size. At step 264, the knee replacement application 40 retrieves data 60 corresponding to the selected femoral implant size. For example, the femoral implant size data 60 may comprise geometrical information corresponding to each available femoral implant such that the knee replacement application 40 may determine the proper guide position and orientation relative to the femur based on the selected implant size. In operation, the guide is attached to the femur and used to perform the posterior femoral resection and to indicate on the femur the location of the keel of the femoral implant.

At step 266, the knee replacement application 40 determines the placement of the femoral implant relative to the femur of the subject. For example, the knee replacement application 40 determines the placement of the femoral implant using the kinematic data 54 acquired at step 238 in combination with the sclerotic bone location data 56 acquired at step 246. The knee replacement application 40 also determine the placement of the femoral implant using information associated with the location of the femoral resection plane determined at step 256. At step 268, the knee replacement application 40 then determines the location and position of the guide relative to the femur corresponding to the implant size. For example, as described above, the knee replacement application 40 evaluates the kinematic data 54 acquired at step 238, the sclerotic bone data 56 acquired at step 246, the femoral resection plane location determined at step 254, and data 60 associated with the particular implant size to locate and position the guide relative to the femur of the subject.

At step 270, the knee replacement application 40 displays on display device 12 the target location and position of the guide, indicated generally by 121, relative to the virtual representation of the selected knee, as best illustrated in FIG. 8. At step 272, the knee replacement application 40 requests placement of the guide 121 relative to the femur. At step 274, the tracking system 22 tracks the guide 121 relative to the subject. For example, as described above, the guide 121 may be coupled or otherwise connected to a trackable element array such that the guide 121 may be tracked using tracking system 22 and calibrated or registered to the subject reference frame. At step 276, the knee replacement application 40 displays the location/position of the tracked guide 121 relative to the target location/position of the guide on the displayed virtual representation of the knee. At decisional step 278, the knee replacement application 40 determines whether the tracked guide 121 is aligned with the target location/position of the guide. If the guide 121 is not properly aligned, the method returns to step 274. If the guide 121 is properly aligned, the method proceeds from step 278 to step 280, where the knee replacement application 40 may signal guide alignment. For example, the knee replacement application 40 may signal alignment using a visible display on display device 12, an audible signal, or other means for indicating to the user the alignment. At step 282, the knee replacement application 40 stores the aligned guide location/position data 62. At step 284, the knee replacement application 40 determines femoral burring surface data 70 corresponding to the femur of the subject. For example, based on the guide alignment data 62, the knee replacement application 40 determines the femoral burring preparation required for the selected femoral implant. Additionally, after alignment of the guide, the guide may be secured to the femur of the subject and the posterior femoral resection may be performed as well as femoral preparation for the keel of the femoral implant.

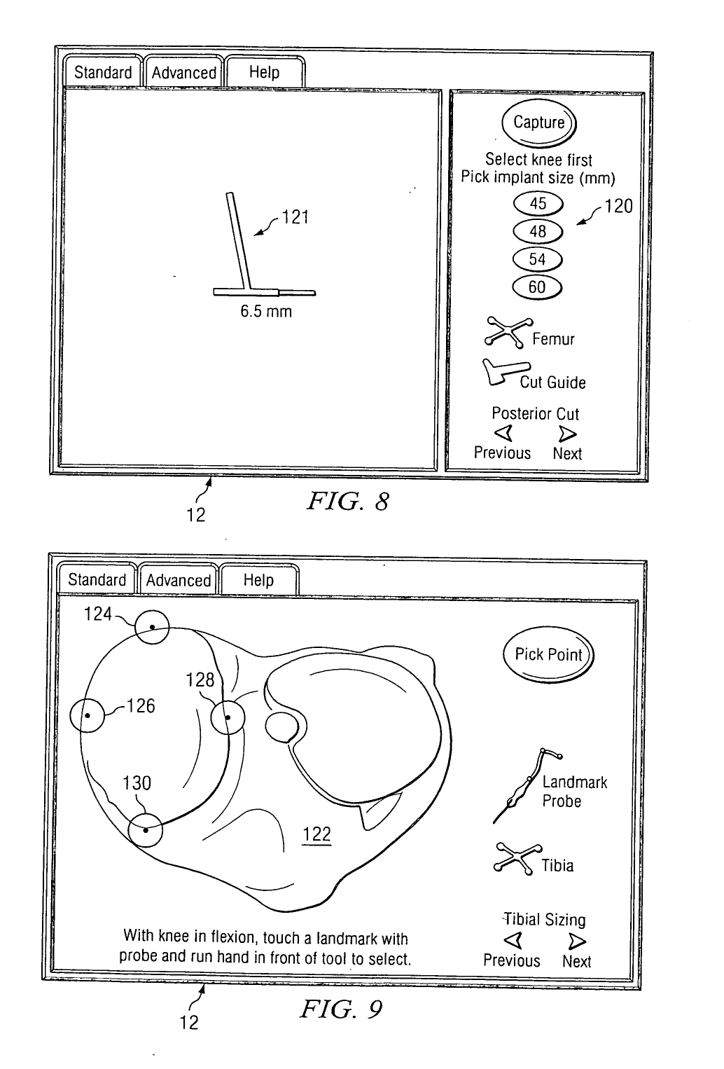

At step 286, the knee replacement application 40 displays a virtual representation 122 of a surface of a tibia on display device 12, as best illustrated in FIG. 9. At step 288, the knee replacement application 40 requests identification of posterior, medial, and anterior border points on the tibial surface. For example, as best illustrated in FIG. 9, the knee replacement application 40 may indicate on the displayed virtual representation 122 of the tibial surface posterior 124, medial 126,128, and anterior 130 border points to be selected by a user using a trackable tool 20. At step 290, the tracking system 22 acquires data 72 corresponding to the posterior, medial, and anterior tibial borders. At step 292, the knee replacement application 40 retrieves implant data 60 corresponding to the tibial implant. For example, the implant data 60 corresponding to the tibial implant may comprise information associated with the various sizes of available tibial implants. At step 294, the knee replacement application 40 determines the tibial implant size based on the acquired posterior/mediaVanterior tibial border data 72 acquired at step 290.

At step 296, the knee replacement application 40 determines the tibial implant position relative to the tibia of the subject. For example, the knee replacement application 40 determines the position of the tibial implant relative to the tibia of the subject based on the tibial border data 72 acquired at step 290.

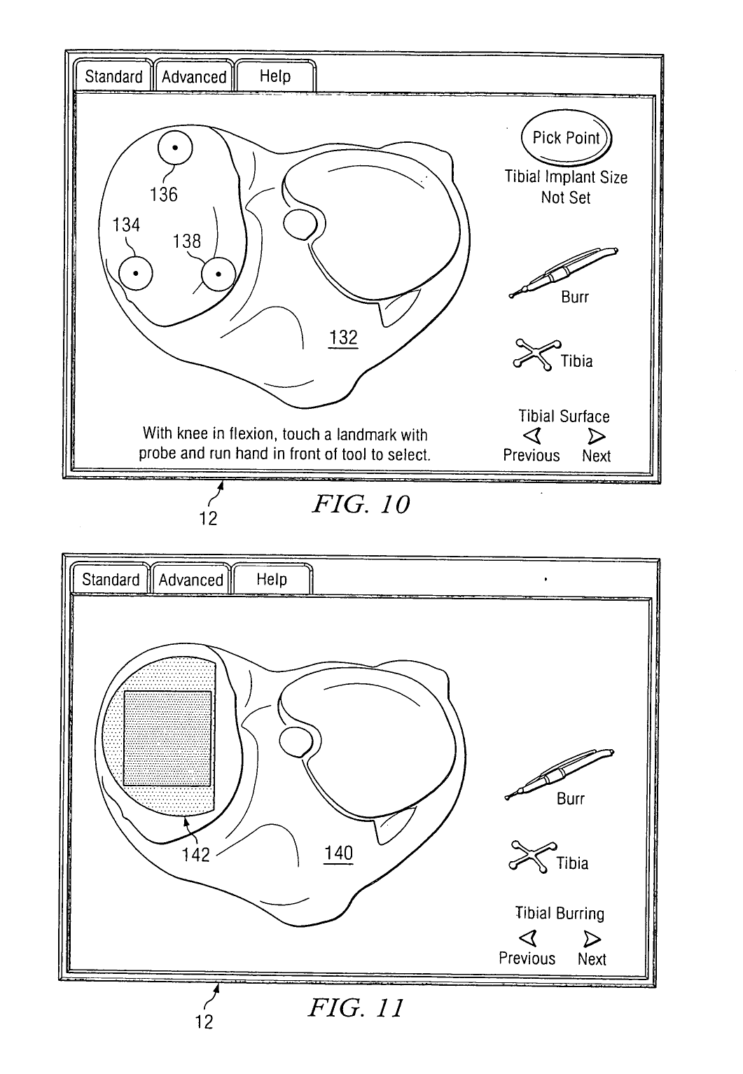

At step 298, the knee replacement application 40 displays a virtual representation 132 of the surface of the tibia on display device 12. At step 300, the knee replacement application 40 requests identification or selection of various locations 134, 136 and/or 138 on the tibial surface, as best illustrated in FIG. 10. For example, as illustrated in FIG. 10, the knee replacement application 40 may indicate various locations 134, 136 and/or 138 on the tibial surface of the displayed virtual representation 132 of the knee for the user to select or identify using a trackable tool 20. At step 302, the tracking system 22 acquires data 50 corresponding to the tibial surface corresponding to the selected points on the tibial surface. At step 304, the knee replacement application 40 determines tibial surface burring data 74 corresponding to the slope and depth of tibial preparation required to accommodate the tibial implant.

At step 306, the knee replacement application 40 displays a virtual representation 140 of the tibial surface on display device 12 with a burring indicator and/or depth guide 142, as best illustrated in FIG. 11. For example, as illustrated in FIG. 11, the knee replacement application 40 displays a virtual representation 140 of the tibial surface to receive burring in preparation for the tibial implant by color coding the virtual representation 140 corresponding to a particular depth and slope corresponding to the selected tibia implant. At step 308, the knee replacement application 40 requests selection of a burring tool 20. At step 310, the tracking system 22 acquires location and positional data of the burring tool 20 relative to the tibial surface of the subject. For example, as described above, a trackable element array may be coupled or otherwise connected to the burring tool 20 such that tracking system 22 may track the location and position of a tip or burring position of the burring tool 20. At step 312, the knee replacement application 40 automatically updates the burring indicator and/or depth guide 142 displayed on display device 12 corresponding to the burring performed to the tibial surface of the subject. For example, during a burring operation of the tibial surface, the tip of the burring tool 20 is tracked using tracking system 22 and correlated to the tibial surface data 74 acquired at step 302 such that changes to the tibial surface of the subject resulting from the burring procedure may be automatically monitored and displayed on display device 12. Therefore, in operation, the knee replacement application 40 provides real-time monitoring of the tibial burring procedure in relation to a target or predetermined tibial burring guide based on the subject's tibia and the selected tibia implant. At decisional step 314,a determination is made whether tibial burring is complete. If tibial burring is not complete, the method returns to step 310. If tibial burring is complete, the method proceeds to step 316.

At step 316, the knee replacement application 40 displays a virtual representation of a femoral surface on display device 12 with a burring indicator and/or depth guide. For example, as described above in connection with the tibial burring procedure, a similar display may be generated by knee replacement application 40 corresponding to femoral burring in preparation for the femoral implant. Thus, at step 318, the knee replacement application 40 requests selection of a trackable burring tool 20. At step 320, the the tracking system 22 acquires location and positional data of the burring tool 20 relative to the femoral surface of the subject. For example, the knee replacement application 40 correlates the location and position of the tip of the trackable burring tool 20 to the femoral surface burring data 70 determined at step 284. For example, based on the location and position of the guide as indicated and stored at step 282, the knee replacement application 40 automatically determines the proper femoral burring preparation for receiving the femoral implant. At step 322, the knee replacement application 40 automatically updates the burring indicator and/or depth guide corresponding to actual femoral surface burring using tracking system 22. For example, as described above, the tracking system 22 automatically tracks the location of the tip of the trackable burring tool 20 relative to the femoral surface during the femoral burring procedure and correlates the actual location of the tip of the trackable burring tool 20 to the target femoral burring preparation surface. At decisional step 324, a determination is made whether femoral surface burring is complete. If femoral surface burring is not complete, the method returns to step 320. If femoral surface burring is complete, the method ends, and the remaining procedure of implanting the tibial and femoral implants into the subject may continue.

What is claimed is:

1. A computer-assisted knee replacement apparatus, comprising:
a storage medium for storing a knee replacement application which, when executed by a processor, displays a series of interface images for assisting a user with a unicondylar knee replacement procedure.

2. The apparatus of claim 1, wherein the knee replacement application is adapted to cooperate with a tracking system to provide real-time knee implant location assistance to the user during the unicondylar knee replacement procedure.

3. The apparatus of claim 1, wherein the knee replacement application is adapted to cooperate with a tracking system to provide real-time knee resection location assistance to the user during the unicondylar knee replacement procedure.

4. The apparatus of claim 1, wherein the knee replacement application is adapted to display a virtual representation of a knee to the user for the unicondylar knee replacement procedure.

5. The apparatus of claim 1, wherein the knee replacement application is adapted to cooperate with a tracking system to acquire kinematic data associated with a tibial sclerotic bone path of a subject knee.

6. The apparatus of claim 5, wherein the knee replacement application is adapted to determine a position for a femoral implant based on the tibial sclerotic bone path.

7. The apparatus of claim 1, wherein the knee replacement application is adapted to cooperate with a tracking system to acquire tibial and femoral anatomical data and determine an extension gap for a subject knee.

8. The apparatus of claim 1, wherein the knee replacement application is adapted to display to the user a plurality of knee implant sizes for the unicondylar knee replacement procedure.

9. The apparatus of claim 1, wherein the knee replacement application is adapted to cooperate with a tracking system to acquire femoral anatomical data and determine a femoral resection plane for the unicondylar knee replacement procedure.

10. The apparatus of claim 9, wherein the knee replacement application is adapted to cooperate with the tracking system to provide real-time alignment data of a resection guide corresponding to the determined femoral resection plane.

11. The apparatus of claim 1, wherein the knee replacement application is adapted to cooperate with a tracking system to acquire tibial anatomical data and determine a tibial resection plane for the unicondylar knee replacement procedure.

12. The apparatus of claim 1, wherein the knee replacement application is adapted to determine a femoral burring requirement corresponding to a particular femoral implant of the unicondylar knee replacement procedure.

13. The apparatus of claim 1, wherein the knee replacement application is adapted to cooperate with a tracking system to display a real-time burring indicator corresponding to an implant burring process of the unicondylar knee replacement procedure.

14. The apparatus of claim 1, wherein the knee replacement application is adapted to cooperate with a tracking system to acquire tibial anatomical data and determine a tibial implant size for a subject knee.

15. The apparatus of claim 1, wherein the knee replacement application is adapted to determine a tibial implant burring requirement corresponding to a particular tibial implant for of the unicondylar knee replacement procedure.

16. The apparatus of claim 1, wherein the knee replacement application is adapted to display an interface image requesting selection of either a right knee or a left knee for the unicondylar knee replacement procedure.

17. The apparatus of claim 1, wherein the knee replacement application is adapted to display an interface image requesting the user to acquire anatomical data corresponding to a designated location on the subject knee.

18. The apparatus of claim 1, wherein the knee replacement application is adapted to display an interface image requesting the user to acquire anatomical data corresponding to a designated location displayed on a virtual representation of a knee.

19. The apparatus of claim 1, wherein the knee replacement application is adapted to display a virtual representation of a subject knee having a burring indicator overlayed thereon to assist the user with a knee burring implant preparation process.

20. A computer-assisted surgery system, comprising:
a display device; and
a knee replacement application executable by a processor and adapted to display on the display device a series of interface images to assist a user with a unicondylar knee replacement procedure.

21. The system of claim 20, wherein the knee replacement application is adapted to cooperate with a tracking system to provide real-time implant location assistance to the user during the unicondylar knee replacement procedure.

22. The system of claim 20, wherein the knee replacement application is adapted to display a virtual representation of a subject knee on the display device for the unicondylar knee replacement procedure.

23. The system of claim 20, wherein the knee replacement application is adapted to cooperate with a tracking system to acquire kinematic data associated with a tibial sclerotic bone path of a subject knee.

24. The system of claim 23, wherein the knee replacement application is adapted to determine a position of a femoral implant based on the tibial sclerotic bone path.

25. The system of claim 20, wherein the knee replacement application is adapted to display to the user a plurality of knee implant sizes for the unicondylar knee replacement procedure.

26. The system of claim 20, wherein the knee replacement application is adapted to cooperate with a tracking system to acquire femoral anatomical data and determine femoral resection data for a femoral implant of the unicondylar knee replacement procedure.

27. The system of claim 26, wherein the knee replacement application is adapted to cooperate with the tracking system to provide real-time alignment data of a resection guide corresponding to the determined femoral resection data.

28. The system of claim 20, wherein the knee replacement application is adapted to cooperate with the tracking system to acquire tibial anatomical data and determine tibial resection data for a tibial implant of the unicondylar knee replacement procedure.

29. The system of claim 20, wherein the knee replacement application is adapted to determine a femoral burring requirement to accommodate a particular femoral implant of the unicondylar knee replacement procedure.

30. The system of claim 20, wherein the knee replacement application is adapted to determine a tibial burring requirement to accommodate a particular tibial implant of the unicondylar knee replacement procedure.

31. The system of claim 20, wherein the knee replacement application is adapted to cooperate with a tracking system to provide a real-time burring indicator corresponding to an implant burring process of the unicondylar knee replacement procedure.

32. The system of claim 20, wherein the knee replacement application is adapted to cooperate with a tracking system to acquire tibial anatomical data and determine a tibial implant size for a subject knee.
